(19) United States
(12) Patent Application Publication
(10) Pub. No.: US 2011/0230899 A1
(43) Pub. Date: Sep. 22, 2011

(54) SURGICAL DEVICE FOR FEEDING AT LEAST ONE SUTURE THREAD THROUGH THE EDGE AREA OF A TISSUE OPENING OF AN INDIVIDUAL AND METHOD FOR OPERATING SUCH A DEVICE

(75) Inventor: Peter Paul Fink, Holzkirchen (DE)

(73) Assignee: MEDI-GLOBE VASCUTEC GMBH, ROHRODORF-ACHENMUEHLE (DE)

(21) Appl. No.: 12/998,782

(22) PCT Filed: Oct. 22, 2010
(86) PCT No.: PCT/DE2010/001243

§ 371 Date: Jun. 1, 2011

(30) Foreign Application Priority Data
Oct. 23, 2009 (DE) 10 2009 050 457.5

(51) Int. Cl. A61B 17/04

(52) U.S. Cl. 606/145

(57) ABSTRACT

In order to guide at least one suture thread (10) attached to a needle (11) through the edge region of a tissue opening and to retract the respective suture thread end from the tissue opening after it has been guided through the edge region of the opening, the surgical device is provided with a rod-shaped needle accommodation and guidance device (5), in which the respective needle (11) is displaceable by means of a pushing device to a needle accommodation region at the distal end of the device by way of an intermediate space region (12), which has a respective length that corresponding at least to the thickness of the wall of the tissue opening, and an outer shape that is reduced relative to the outer shape of the needle accommodation and guidance device in order to allow unhindered movement of the respective needle (11) from the needle accommodation and needle guidance device (5) to the distal end of the device, the device being further provided with a needle accommodation region on its distal end, having a mesh (9) which is compressible or extendable in the longitudinal direction of the device and in which the respective needle (11) is retained permanently after having been guided through the edge region of the tissue opening.

The invention relates to a surgical device for guiding at least one suture thread, attached to a suturing needle, through the edge region of a tissue opening,

particularly a tissue opening present in the artery of an individual, optionally formed by incision, and for retracting the suture thread end guided through the edge region in question from said opening,
having a rod-shaped needle accommodation and guidance device, in which the needle attached to the respective suture thread is accommodated by and guided in an accommodation and guidance opening such that it is movable from a pushing device disposed at the proximal end of the device to a needle accommodation region at the distal end of the device, by way of an intermediate space region whose length corresponds at least to the thickness of the wall of the tissue opening, and having an outer shape that is reduced relative to the outer shape of the needle accommodation and guidance device in order to allow unhindered movement of the needle in question from the needle accommodation and guidance device to the distal end of the device,
the respective needle being movable to the needle accommodation region, for permanent accommodation there, after having been guided through the edge region of the tissue opening in question,
and the rod-shaped needle accommodation and guidance device, together with the respective needle permanently accommodated in the needle accommodation region, and the suture thread end connected thereto then being retractable from the above-mentioned tissue opening. The invention further relates to a method for operating such a device.

A device for guiding at least two suture threads through the wall of a sheath, a balloon, or a surface, particularly of an artery of an individual, near the edge region of an opening present therein, optionally formed by incision and/or dissection, and for retracting the suture thread ends guided through the edge region in question from said opening is already known (DE 199 42 951). In this known device, a rod-shaped thread guidance device is provided, in which the suture threads attached to needles are guided in guidance or accommodation openings such that they are capable of being moved by means of the thread guidance device near the edge region of the respective opening through said wall and of being retracted from the opening such that the opening can be closed by means of pulling together and, optionally, knotting the suture thread ends outside of the respective opening.

In this known device, the thread guidance device has in its longitudinal direction a proximal thread feeding part, a distal thread accommodation part, and a central thread release/thread clamping part located therebetween. Said central thread release/thread clamping part is rotatable at least relative to the distal thread accommodation part and has a cross-section designed to allow, at least in one rotational position, the insertion of the suture threads fed from the suture thread feeding part provided at the proximal end of the device into accommodation openings exposed in the thread accommodation part and to allow, in rotational positions other than the above mentioned rotational position, the suture threads accommodated in the respective accommodation openings to be gripped along with the needles, in order to enable the retraction of the entire thread guidance device from the aforementioned opening.

Although this known device allows the satisfactory closure of openings in the wall of a sheath, particularly ones in an artery wall of an individual, it is still desirable to provide a surgical device for guiding at least one suture thread through the edge region of a tissue opening that is easier to operate and eliminates rotational movements of individual parts of the device.

Such a surgical device that is simpler to operate is known from DE 10 2004 041 936 B3. In this known surgical device, the needle dispensing region, the intermediate space region and the needle accommodation region of the rod-shaped needle and thread guidance device are rigidly interconnected. In addition, the rod-shaped needle and thread guidance device may be closed by an elongated external member surrounding its intermediate space region with a positive fit, the elongated external member being movable relative to the rod-shaped thread guidance device in its longitudinal direction while releasing the above-mentioned intermediate region. Furthermore, the needle dispensing region has an end sides toward the distal end of the device and the elongated external member has an end side in its area toward the proximal end of the device, said end sides facing each other.

Although this known surgical device is easier to operate than the known device discussed at the outset, it is still desirable to simplify the construction of a surgical device for guiding at least one suture thread through the edge region of a tissue opening even further.

A device for closing an opening in a tissue membrane inside a human body is already known (cf., e.g., U.S. Pat. No. 5,304,184, DE 693 32 912 T2), in which a suture material feeding device containing at least two suture threads and a suture material accommodation device are provided. The suture material feeding device contains suture needles with offset needle tip parts which are initially in a folded state in the radial direction, each containing suture material received from a needle's eye. The suture material accommodation device comprises an expandable, balloon-like structure made of elements capable of arch-shaped bulging, or it is made of an elastomer.

Following the introduction of the suture material accommodation device into the tissue opening to be closed, the balloon-like structure will be expanded such that it can no longer be retracted from the tissue opening in this state. Then, suture needles of the suture material feeding device, with their offset needle tip parts, each containing suture material accommodated in a needle's eye, are pivoted, using a crank-like movement, from a folded position into an external position in the radial direction, in which they are inserted at a distance from the edge region of the tissue opening through the tissue into the balloon-like structure. In this balloon-like structure, only the suture material is gripped in that the balloon-like structure of the suture material accommodation device is returned into its folded state. The suture needles themselves, however, must then be pulled back from or pulled off of the suture material. If they were to remain in the balloon-like structure and if the suture material accommodation device were then to be retracted from the tissue opening, the tissue between its puncture sites and the tissue opening would tear.

Although the use of other needle designs, such as a hollow needle cannula, was also mentioned in conjunction with the known device most recently discussed above, it is not known in this context how the suture material feeding device and the suture material accommodation device would be constructed in the case of such other needle designs. In addition, such a hollow needle cannula would also have to be retracted from the suture material remaining in the balloon-like structure before the suture material accommodation device is retracted with the suture material through the suture opening in order to prevent tearing of the tissue between its puncture sites and the tissue opening.

Moreover, an additional suture device is known (WO 99/012480 A1, WO 99/025254 A1) that also involves the use of hollow needles through which the suture material is guided in one embodiment. Along with the suture material they accommodate, these hollow needles are captured along with the suture material, after penetrating the tissue edge region of a tissue opening to be closed, by a structure such as a balloon provided on the distal end of the suture device and expanded subsequent to its introduction into the tissue opening. The hollow needles are then retracted from the respective expanded structure back to the proximal end of the device, while the suture material ends are gripped in the structure, which is then folded up again. This folded structure, along with the suture material retained thereby, is subsequently retracted from the tissue opening in the direction of the proximal end of the device. The respective tissue opening may then be closed by pulling together and knotting the suture material running through the tissue edge region of the tissue opening to be closed and through the tissue opening itself.

In this known suture device as well, the hollow needles must be pulled off of the suture material and retracted from the tissue opening to be closed before retracting the structure from the tissue opening that is expandable to accommodate the suture material and may be folded back together after accommodating the suture material. Otherwise the tissue between the puncture sites of the hollow needles through the edge region of the tissue opening and the tissue opening would tear in this case as well.

Devices for closing wounds are also known (DE 696 37 177 T2, DE 697 29 920 T2). On their respective distal device end to be introduced into a wound or tissue opening, these devices have a thread accommodation/thread spreading part by means of which the ends of two suture threads are initially held. By means of an adjusting device provided in the proximal device region, the thread accommodation/thread spreading part is movable between a folded position and a spread position. In the folded position, the thread accommodation/thread spreading part may be easily introduced into the wound or tissue opening to be closed. After the subsequent spreading inside the respective wound or tissue opening, suture needles provided with barbed hooks are guided through the edge region of the wound or tissue opening to be closed up to a point where these barbed hooks reach the capture or gripping region of the now-tensioned suture threads accommodated by the thread accommodation/thread spreading part. By subsequently retracting the suture needles in the direction of the proximal device region, the tensioned suture threads are captured by the barbed hooks of the suture needles and pulled through the puncture sites previously created by the suture needles around the wound or tissue opening to be closed. The respective tissue opening may then be closed by pulling together and knotting the suture threads extending through the tissue edge region of the tissue opening to be closed and through the tissue opening itself.

However, the design effort required for the implementation of the known devices for closing wounds discussed above is considered to be relatively complex and costly, in particular in view of the thread accommodation/spreading part and the suture needles equipped with barbed hooks. Furthermore, the mechanism for capturing the suture thread ends accommodated by the thread accommodation/spreading part using the barbed hooks of the suture needles requires a relatively precise control of the respective suture thread tension to ensure that the respective suture thread ends can be captured by the barbed hooks of the suture needles.

Another known suture device (cf., e.g., U.S. Pat. No. 5,417,699, DE 693 34 017 T2) has a needle carrier to be introduced into a tissue opening to be closed as well as a needle accommodation device positioned outside the respective opening. The needle carrier has two bent needles which are connected on one of their ends to the two ends of a suture thread. On their other ends, the needles are provided with tips and the region of the tips is designed with barbed hooks. The needle accommodation device comprises an expandable cage structure.

During operation, the needle carrier is initially advanced completely through the tissue opening of the blood vessel to be closed and into the blood vessel. Then the needles are guided, while radially expanding, through the edge region of the tissue opening in question from the interior of the blood vessel containing the tissue opening and outward through the vessel wall surrounding the tissue opening. In this process, the remaining needle carrier parts initially continue to remain within in the blood vessel. However, these needle carrier parts, together with the suture threads connected thereto, are then pulled out of the blood vessel through the tissue opening so that only the part of the suture thread connected to the already-retracted needles remains behind in the blood vessel. The needles, along with their barbed hooks, are advanced into the cage structure now expanded outside the tissue opening to be closed and retained due to its subsequent contraction. The subsequent retraction of the contracted cage structure with the needles held tightly therein causes the suture thread portion connected to these needles on its ends to be tensioned inside the above-mentioned blood vessel, allowing the suture thread ends to then be tied into knots. This allows the respective opening to be closed.

In principle, although the known device considered above does permit suture threads to be guided through the wall of a blood vessel near the edge region of an opening present therein, safety is critical at least with regard to the accommodation of the needle ends in the needle accommodation device. This is because, if one needle or another is not securely accommodated by the needle accommodation device, complications cannot be ruled out and additional interventions may be necessary, which is considered undesirable. For reasons of suited dimensions, the access to the blood vessel, specifically to the artery, must also be widened in this case. This, however, complicates a minimally invasive use of the known device in question.

The object of the invention is therefore to refine a device and a method of the type stated at the outset, such that both a simpler and a more secure accommodation of at least one suture needle guided through the tissue edge of a tissue opening and carrying a suture thread is made possible.

The object defined above is attained according to the invention by a surgical device of the type stated at the outset in that the needle accommodation region of the rod-shaped needle accommodation and guidance device is formed by a mesh which is attached at the distal end of the device and is capable of being compressed around a longitudinal body component in a longitudinal direction of the device and of being extended in an opposite longitudinal direction of the device, said mesh, in its compressed state, allowing the respective needle to be accommodated in an interior space of the mesh created by the compression and enclosing and gripping only the needle in question after the accommodation of such a needle by its subsequent extension.

This invention has the advantage that the respective suture needle is capable of being accommodated and retained, following its puncture of the edge region of a tissue opening, in a simpler yet secure way by the needle accommodation and guidance device as compared to the known suture devices discussed above. The retention of the respective needle is effected preferably by positive form locking produced by restretching the mesh following the previously accomplished needle accommodation. Thus, the invention is advantageously suitable for minimally invasive use for closing a tissue opening of an individual.

It is expedient for the respective suture thread to run from a fixed device handle part within a single suture thread channel, particularly situated near the exterior of the device, to the distal end region of the needle accommodation and guidance device and from there to its associated needle. This produces the advantage of a simple and safe management of the respective suture thread in the event of its separation from the associated suturing needle, which continues to be retained in said mesh interior space by a positive fit.

Preferably, the needle accommodation and guidance device has a separate guidance channel for each needle, said guidance channel being provided with an entry opening near its distal end, at least for the insertion of the suture thread connected to the respective needle. This measure facilitates both the introduction of the respective suturing needle with its suture thread into the needle accommodation and guidance device and the management of the respective suture thread in the event of its detachment from the associated suture needle gripped by the aforementioned mesh interior space by positive shape fit.

In another expedient embodiment of the invention, the respective suturing needle can be displaced from a position adjacent to the fixed device handle part to a position in the direction of the distal end of the device by means of a needle pushing device, which is displaceable particularly on the needle accommodation and guidance device. This embodiment offers the advantage of a displaceability of the respective suturing needle that is particularly easy to implement in terms of its design.

Yet another embodiment of the invention has the particular advantage that the displaceability of the respective needle may be released only if the mesh is compressed, forming the mesh interior space. Thus, the respective suturing needle cannot be displaced until it can be positively accommodated and held tightly by the mesh. The risk of injury while handling the surgical device according this embodiment prior to its use for guiding at least one suture thread through the edge region of a tissue opening, particularly one present in an artery of an individual, is virtually eliminated.

It is practical to provide a release/blocking mechanism for releasing or blocking the displacement of the respective needle between the fixed device handle part and a rotation device adjacent thereto. The use of such a release/blocking mechanism has the advantage of safely blocking of the displaceability of the needle in question.

According to an expedient refinement of the invention, the release/blocking mechanism has a male part with a specific outer shape and a female part with an internal shape matching the respective outer shape which allows the male part to be accommodated in a fixed rotational position of the male part relative to the female part, the male part or the female part being connected to the rotation device and the female part or the male part being connected to the fixed device handle part. This provides the advantage of a release/blocking mechanism based on the principle of keyhole and matching key, which may be realized with particular ease.

In the release/blocking mechanism considered above, the rotation device is preferably connected to the fixed device handle part by a conversion device translating a rotational movement to a translational movement, said conversion device having a pin device, rotating along with the rotation of the rotation device about the circumferential direction of the latter, and a pushing part translating its rotational movement into a longitudinal movement, said pushing part making the mesh compressible, thus forming its mesh internal space. This construction has the advantage that this release/blocking mechanism can be implemented with particularly little design effort.

It is expedient to position the rotation device on the proximal end of the device adjacent to the fixed device handle part and to position the pushing member adjacent to the fixed device handle portion in the direction of the distal end of the device. This results in the advantage of allowing the user to hold the surgical device according to this embodiment in one hand and conveniently operate it with the other hand.

The object stated above is additionally attained by a method of operating a surgical device according to the invention for guiding at least one suture thread attached to a suturing needle through the edge region of a tissue opening present particularly in an artery of an individual and optionally created by incision, and for retracting the respective suture thread end from the above-mentioned opening after it was guided through the edge region, with the needle connected to the respective suture thread being accommodated and guided in an accommodation and guidance opening of a rod-shaped needle accommodation and guidance device such that it is displaced by a pushing device arranged in the direction of the proximal end of the device toward a needle accommodation region at the distal end of the device via an intermediate space region, the length of which corresponds at least to the thickness of the wall of the tissue opening and having an outer shape, that is reduced relative to the outer shape of the needle accommodation and guidance device, in order to allow unhindered movement of the needle in question from the needle accommodation and guidance device to the distal end of the device, and with the respective needle being movable to the needle accommodation region for permanent accommodation there after having been guided through the edge region of the tissue opening in question, and with the rod-shaped needle accommodation and guidance device, together with the needle permanently accommodated in the needle accommodation region, and the suture thread ends connected thereto, then being retractable from the above-mentioned tissue opening.

This method is characterized in that the respective suturing needle accommodated in the needle accommodation region of the rod-shaped needle accommodation and guidance device is pushed through the mesh attached to the distal end of the device and compressible about a longitudinal body component in a longitudinal direction of the device and extendable in an opposite longitudinal direction of the device, with the mesh in its compressed state, into a mesh internal space produced by the compression, and is enclosed and retained by the mesh after such needle accommodation by the subsequent extension of the mesh.

This produces the advantage of easy, yet safe handling of the surgical device according to the invention. This handling preferably allows one-handed operation of the device in question, whereby its re-extended mesh preferably holds the respective needle in this mesh in a positive fashion with a shape fit subsequent to the needle accommodation.

The invention will be explained in greater detail below using an exemplary embodiment with reference to the drawings.

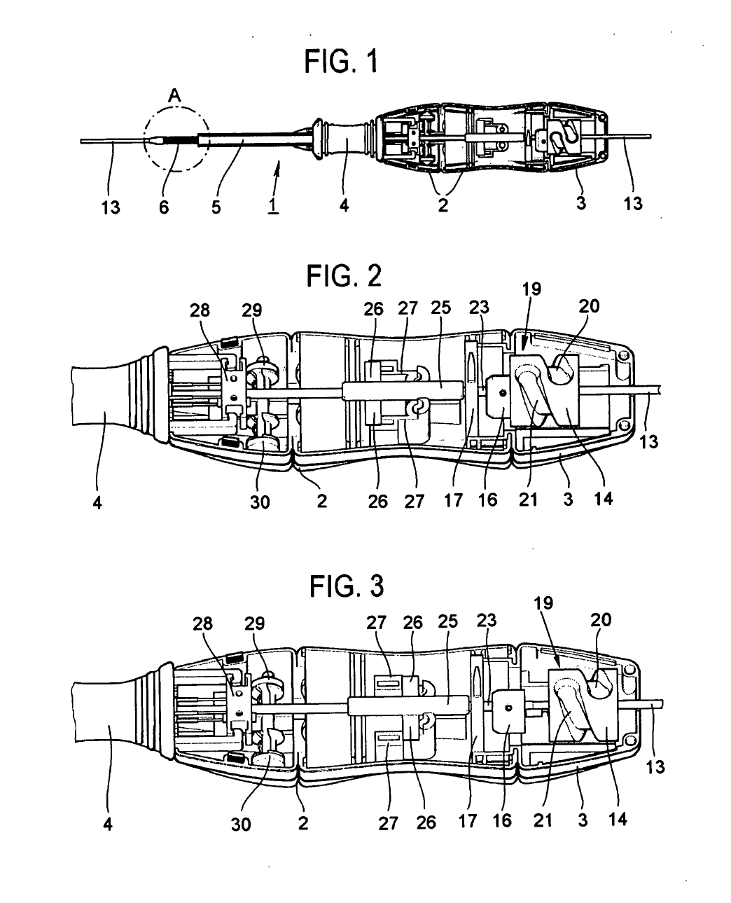

FIG. 1 shows, in a schematic top view not drawn to scale, a surgical device according to an embodiment of the present invention in a partially opened state in a first position with a pushing part being in the first position.

FIG. 2 shows, in a slightly enlarged top view, the largely open proximal region of the device shown in FIG. 1 with the pushing part in the first position.

FIG. 3 shows, in a slightly enlarged top view, the largely open proximal region of the device shown in FIG. 1 with the pushing part in a second position.

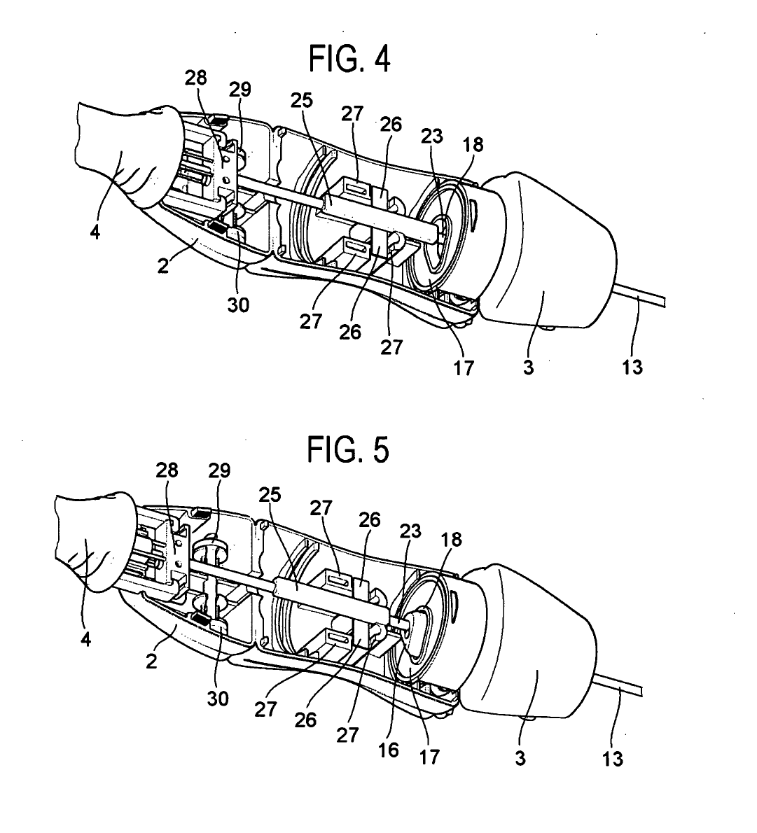

FIG. 4 shows, in a perspective view, the opened portion of the device according to FIG. 1 shown in FIG. 3, with a needle pushing device in a first position.

FIG. 5 shows, in a perspective view, the opened portion of the device according to FIG. 1 shown in FIG. 3, with the needle pushing device in a second position different from the first position.

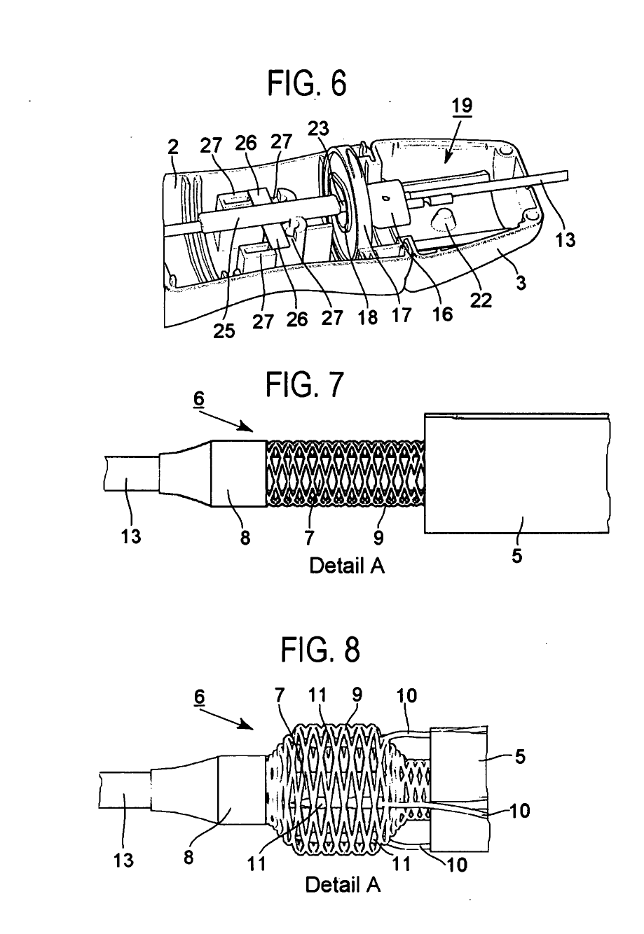

FIG. 6 shows, in a perspective view, the embodiment of a rotation part on the opened proximal end of the device, with the pushing part removed.

FIG. 7 shows, in an enlarged top view, the detail of the highlighted portion A of the device according to FIG. 1 as a mesh belonging to a needle accommodation region in its extended state on the distal end of the device.

FIG. 8 shows, in an enlarged top view, the detail of the highlighted portion A in the device according to FIG. 1 as a mesh belonging to the needle accommodation region in its compressed state on the distal end of the device, forming a mesh interior space in which needles containing suture threads may be or are accommodated in the second position of the pushing part for the purpose of being gripped thereafter.

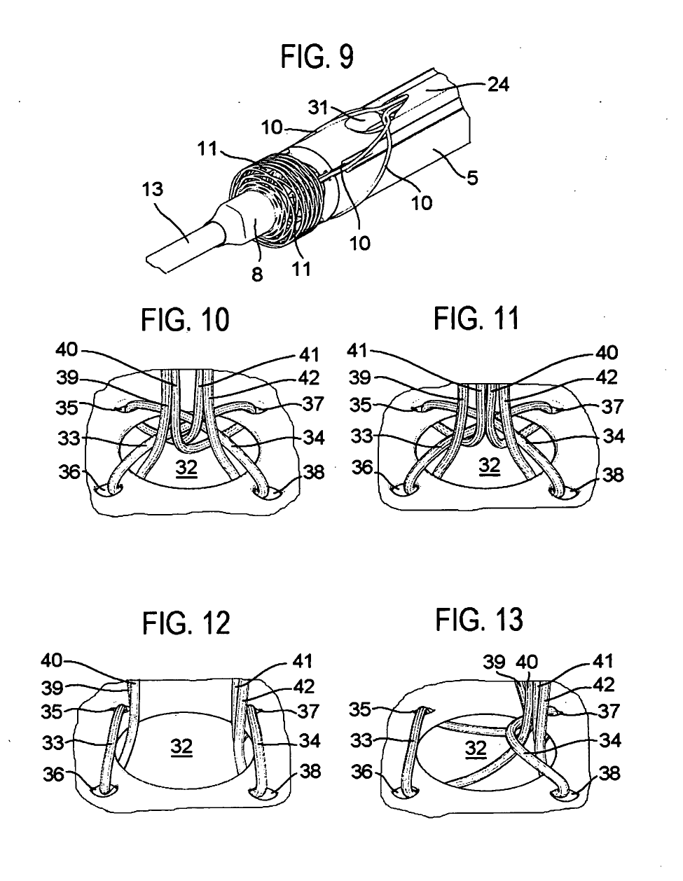

FIG. 9 shows, in an enlarged perspective view, the highlighted portion A of the mesh of the device according to FIG. 1, in its compressed state on the distal end of the device, along with the needles accommodated by the mesh, showing the path of the suture threads connected to the needles.

FIG. 10 to FIG. 13 show, in greatly enlarged perspective views, the path of suture threads which have been guided by means of four needles through the edge region of a tissue opening to be closed and then retracted from the tissue opening, along with the suture thread ends connected to the needles, using the device according to FIG. 1.

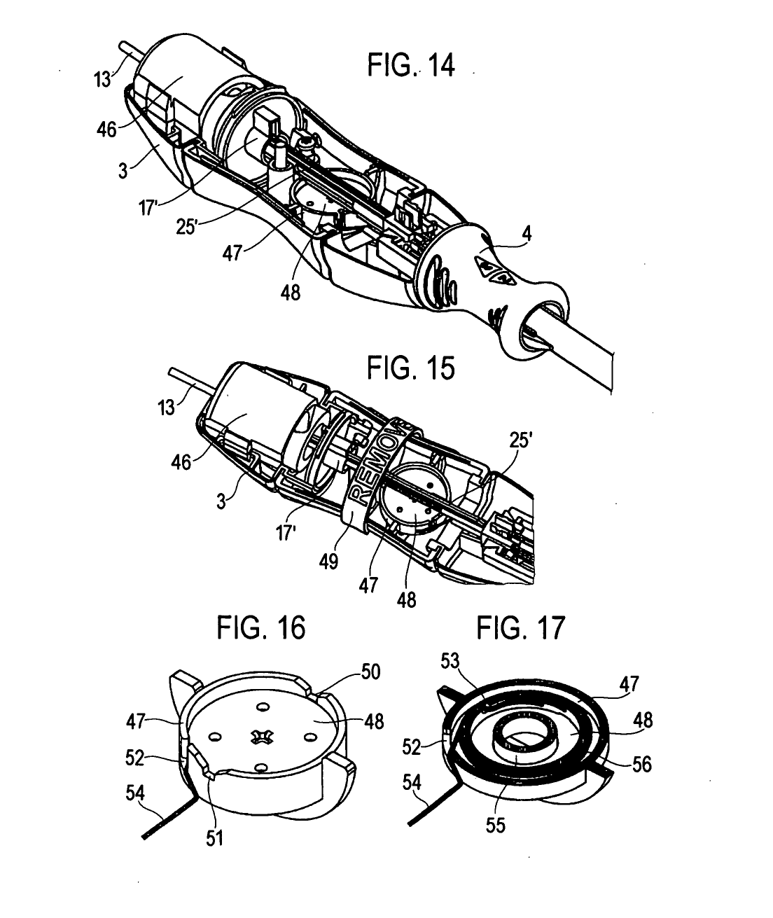

FIG. 14 shows the largely open proximal area of this device in a perspective view with a needle pushing device for a further embodiment of the surgical device in accordance with the present invention.

FIG. 15 shows a perspective view of a section of the further embodiment of the surgical device according to the invention shown in FIG. 14, along with a removable locking or blocking member.

FIG. 16 shows, in a considerably enlarged perspective view, a thread take-up device with a thread spool and a spool receiving body receiving it, said thread take-up device being installed in the further embodiment of the surgical device in accordance with the present invention, as shown in FIGS. 14 and 15.

FIG. 17 shows a perspective sectional view of the thread take-up device shown in FIG. 16 with a section below an upper cover plate of the thread spool.

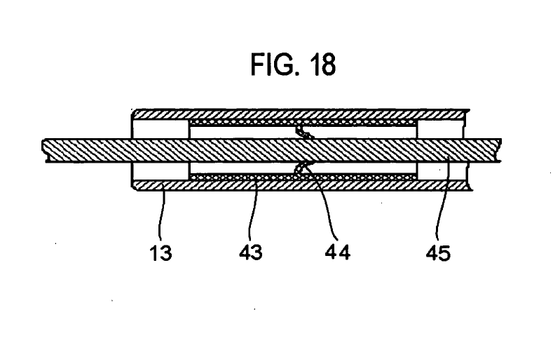

FIG. 18 shows a greatly enlarged sectional view of the distal end region of the device shown in FIG. 1.

Before discussing the drawings in greater detail, it should be noted that the same elements or devices are identified by the same reference characters in all drawings.

FIG. 1 shows a surgical device 1 according to one embodiment of the present invention in a schematic top view not drawn to scale. In this figure, the right-hand portion of device 1 shown in FIG. 1 illustrates a half-shell of device 1, i.e., device 1 in an opened state. In this area, device 1 preferably comprises two half-shells that may be assembled by interlocking and/or screwing together to form a closed housing part.

The housing part of surgical device 1 comprises a fixed handle part 2 shown in the central region of FIG. 1, linked to a rotation part 3 on the proximal end region shown on the right side and a needle pusher 4 on the distal end region shown on the left side, said needle pusher constituting a needle pushing device. As will become more evident further below, this needle pusher 4 serves to displace suture needles not shown in FIG. 1 and simply referred to as needles in the following. These needles, along with the ends of suture threads connected thereto, are housed in the distal end region of a rod-shaped or tubular needle accommodation and guidance device 5, which connects to the needle pusher 4 in the direction of the distal end of the device. In a practical embodiment with tubular design, this needle accommodation and guidance device 5 has an outer diameter of, e.g., no more than 20 FR (French), i.e., approx. 6.7 mm.

As may be seen in greater detail in FIGS. 7, 8, and 9, at the distal end of the device, needle accommodation and guidance device 5 is adjoined by a needle accommodation device 6, which has a longitudinal body component 7, shown in FIGS. 7 and 8, which is displaceable in the longitudinal direction of device 1, as well as a distal end part 8 having a shape that is conical towards the distal end, and a mesh 9 that may be compressed in the longitudinal direction of device 1 and extended in an opposite longitudinal direction of the device. As shown in greater detail in FIGS. 7, 8, and 9, this mesh 9, which is located in a separately marked portion A, comprises thin wires or strands, extending in a transverse fashion, preferably at a relatively acute angle, perpendicularly to the longitudinal axis of device 1, similar to an electrical shielding mesh of a coaxial antenna cable. The preferred material for the thin wires or strands is stainless steel or a different biocompatible mesh material whose shape can be changed from an extended shape to a compressed shape, in which it is expanded transversely to the longitudinal axis of device 1 and forms a mesh interior space. Mesh 9 is close-meshed in the extended state and wide-meshed in the compressed and hence expanded state.

To increase the reliability of catching the needles to be accommodated and gripped by mesh 9 and to prevent needles from escaping after having been accommodated, mesh 9 may be coated with an anti-slip material, such as an elastomer, preferably at least on its side facing the longitudinal body component 7. However, it is also conceivable for the wires or strands of mesh 9 themselves to be made from such an anti-slip material, such as an elastomer.

With respect to longitudinal body component 7 and its distal end part 8, it should be noted that longitudinal body component 7 has a reduced outer dimension as compared to the outer dimension of distal end part 8; given a round design of these parts 7, 8, these outer dimensions are their outer diameter. This allows the needles 11 to be enclosed and retained in the region of the longitudinal body component 7 within the close-meshed mesh 9, as shown in FIG. 7. Here, the respective mesh 9 is rigidly connected to distal end part 8 and the distal end of the needle accommodation and guidance device 5, respectively.

The expanded mesh interior space with the now wide-meshed mesh 9 allows the accommodation of needles 11, connected to the suture thread ends 10, as shown in FIGS. 8 and 9. When mesh 9 is subsequently stretched, it again assumes its shape shown in FIG. 7, i.e., it is extended again. In this extended shape, needles 11 are now enclosed and therefore positively held by mesh 9. Needles 11 can then no longer be pulled out of mesh 9 by exerting a pulling force by means of the connected suture thread ends or suture threads 10.

In a practical embodiment and in its extended state, given a tubular design, mesh 9 has an outer diameter of, e.g., no more than 12 FR, i.e., 4 mm.

With respect to FIGS. 8 and 9, it should also be noted that the needles 11 can be pushed out of edge-adjacent needle dispensing regions of the needle accommodation and guidance device 5. These edge-adjacent needle dispensing regions of needle accommodation and guidance device 5 define an intermediate space region 12 in conjunction with the region of the non-compressed mesh 9. This intermediate space region 12 has a length that corresponds at least to the thickness of the wall of the tissue opening. On the other hand, the intermediate space region 12 has an outer shape, relative to the outer shape of needle accommodation and guidance device 5, that is reduced to allow unhindered movement of the respective needle 11 from the needle accommodation and guidance device 5 to the distal end of the device. This means that the respective needle 11 may be guided, along with the attached suture thread end 10, through the edge region of the tissue opening in question at some distance from the edge.

Returning to FIG. 1, it should be noted that it also shows an insertion tube 13, particularly made of plastic, metal or a composite material, which extends through device 1. On the one hand, this insertion tube 13, in conjunction with end part 8, which has a conical shape towards the distal end, facilitates the introduction of the distal end of device 1 into a tissue opening to be closed of an individual. On the other hand, the respective insertion tube 13 facilitates the accommodation of a guide wire, shown in FIG. 14, from the proximal end of device 1, said guide wire then being left in the tissue containing the tissue opening to be closed, before at least one needle 11, connected to an end of suture thread 10, is guided through the edge regions of this tissue opening by means of device 1. Preferably, however, a plurality of needles 11, for example four needles 11, which are respectively connected to an end of a suture thread 10, are guided through the edge regions of the tissue opening. Insertion tube 13 is positioned immovably, particularly in the longitudinal center of device 1. When using an even number of needles, the center-to-center distance between the diagonally opposite needles relative to the tissue opening to be closed is preferably 16 FR (i.e., 5.3 mm).

On the aforementioned insertion tube 13, which is preferably made of a flexible plastic tube, such as a polyamide, in the distal end region of device 1 and of a metal tube in the proximal end region of device 1, the aforementioned distal end part 8 may be rigidly mounted, with the longitudinal body component 7 possibly formed by a section of insertion tube 13. By using the flexible plastic tube in the distal end region of device 1, the latter is flexible in that area when introducing the respective insertion tube 13 into a tissue opening to be closed.

FIG. 2 illustrates the largely open proximal region of device 1 in a slightly enlarged top view and with a pushing part 14 in the same position as in FIG. 1. Rotation part 3 houses a male part 16 having a specific outer shape and belonging to a release/blocking mechanism 15 for the needle displacement as well as a female part 17 in the shape of a revolving disk having such a specific inner shape or accommodation opening that it allows accommodation of the outer shape in a fixed relative rotational position of male part 16 to female part 17 or revolving disk, as is evident from the perspective view shown in FIG. 5. The release/blocking mechanism 15 therefore corresponds to the principle of an ordinary key and matching keyhole of a lock.

Here, the female part 17 or the revolving disk as a rotary part is connected to the rotational member 3, and the male part 16, as will be explained in greater detail later, in the fixed handle part 2 is capable of being displaced only in the longitudinal direction of device 1 by means of a hollow tube 23 connected thereto, a further hollow tube 25 connected thereto, and a coupling mechanism 28 connected to the needle pusher 4 when the outer shape of male part 16 and the accommodation opening of female part 17 or the revolving disk are in congruent positions. Here, male part 16, the aforementioned hollow tubes 23, 25, the coupling mechanism 28 and needle pusher 4 are rigidly connected to each other. It should be noted, though, that female part 17 could, in principle, also be arranged in a non-rotational manner and male part 16 in a rotatable manner. The important point is that a relative rotation between male part 16 and female part 17 or the revolving part be enabled.

In rotation part 3, a movement conversion device 19 is provided in addition to the release/blocking mechanism 15 just considered, which converts a rotational movement of rotation part 3 into a translational movement. This movement conversion device 19 features the aforementioned pushing part 14, which is provided, in this instance, as a cylindrical part, in the cylindrical outer wall of which at least one thread 20 is provided which extends over a fixed angular range, e.g., 90° or 180°, around the respective cylindrical outer wall. In the present case, a second respective thread 21, running in a parallel manner, is provided in addition to thread 20. In this case, the respective thread 20, 21 preferably has a quadrangular, e.g., trapezoidal, cross-section. But the cross-section could also have a different, e.g., triangular, shape.

A pin device 22 engages in the respective thread 20, 21 of pushing part 14 of the movement conversion device 19, said pin device being formed in this case by a pin element 22 protruding from the inner circumference of rotation part 3. This pin element 22 protrudes inward from the inner surface of the respective shell half of rotation part 3, as is clearly evident from FIG. 6. The respective pin element 22 has an external cross-sectional shape that matches the internal cross-sectional shape of the respective thread 20, 21. This causes a rotation of rotation part 3 to produce an axial displacement of pushing part 14.

In this context, it should be noted that, in principle, only one thread 20 or 21 in pushing part 14 and one pin element 22 in one of the shell halves of rotation part 3 is adequate for the movement conversion device 19. However, due to the symmetrical arrangement of two pin elements 22—i.e., one pin element 22 in each shell half of rotation part 3—and the accommodation of one pin element 22 by thread 20 and the other pin element 22 by the other thread 21, a movement conversion device 19 is provided that has a particularly simple design and can be easily actuated, i.e., that functions without jamming.

Insertion tube 13 considered above is connected in a mechanically rigid manner to pushing part 14. As mentioned above, insertion tube 13 extends through device 1 up to its distal end. By displacing this insertion tube 13 to the proximal end of device 1 as a result of a respective displacement of pushing part 14 into the position shown in FIG. 3, mesh 9 is moved from the extended position shown in FIG. 7 to the compressed position shown in FIGS. 8 and 9 by application of a pulling movement to the end part 8 connected to the distal end of the mesh. In this compressed position of mesh 9, it is possible to introduce needles 11 through its meshes into the mesh interior space thus formed, as is illustrated in FIGS. 8 and 9. These needles 11 are connected to the suture thread ends 10, which extend from the fixed device handle part 2 in a single thread or suture thread channel 24, particularly one present near the outside of the device, to an exit opening 31 in the distal end region of needle accommodation and guidance device 5 and from there to their respectively associated needles 11. Suture thread channel 24 is positioned in this case in the outer peripheral region of needle accommodation and guidance device 5 between two needle accommodation openings.

It should be noted in this context, that, in principle, the needle accommodation and guidance channels associated with the individual needles 11 can also be shared to accommodate the ends of suture threads 10 connected to the respective needles 11, i.e., they can be used for a combination of needle and suture thread.

FIG. 3 illustrates the situation of pushing part 14 after the completed rotation of rotation part 3 by the fixed angular value, e.g., 90° or 180°. In this position, needle pusher 4 can be advanced towards the distal end of device 1, as can be seen from FIG. 5 in comparison with FIG. 4. Male part 16 is in the same position relative to female part 17. However, this female part 17 is now rotated relative to male part 16 such that male part 16 can now be received by female part 17 in a sliding fashion or, more precisely, by the opening of such female part, by displacement toward the distal end of the device. This is clearly evident from the perspective views shown in FIGS. 4 and 5 in comparison with FIG. 6.

The displacement of male part 16 in the opening of female part 17, mechanically coupled with rotation part 3, is effected in that a hollow tube 25, mechanically coupled with male part 16, surrounds or is connected to the aforementioned hollow tube 23 and is displaced towards the distal end of the device. This is evident from a comparison of the perspective views shown in FIGS. 4 and 5. As a result, the hollow tube 25 is also displaceable relative to insertion tube 13.

In order to ensure that insertion tube 13 does not perform a twisting motion during the rotation of rotation part 3, insertion tube 13 may be provided with small guide plates 26 sliding in lateral longitudinal slots of hollow tube 25, said small guide plates being guided between the support and guide posts 27 provided in the aforementioned half-shells. This ensures that insertion tube 13 and hollow tube 25 can only be moved in their longitudinal directions, but not turned about their longitudinal axes. This prevents twisting of mesh 9. It should be noted that, in principle, device 1 according to the invention can also do without the aforementioned small guide plates 26.

On its distal end, hollow tube 25 is connected to needle pusher 4 via a coupling mechanism 28. This needle pusher 4 can displace the needles 11 connected to the ends of suture thread 10 in the rod-shaped needle accommodation and guidance device 5 to the distal end of device 1 when rotation part 3 is in the rotational position shown in FIGS. 4 and 5. In this rotational position, pushing part 14 is in the rotational position shown in FIG. 3, which is the position of mesh 9 in its compressed state, as can be seen from FIGS. 8 and 9.

The combined use of release/blocking mechanism 15 with male part 16 and female part 17 on the one hand, and movement conversion device 19 with pushing part 14, which has at least one thread 20 or 21 in its cylindrical circumference, and the pin element 13 associated with the respective thread 20 or 21, ensures that the respective needle 11 cannot be retracted from needle accommodation and guidance device 5 until the point when mesh 9 is compressed for accommodation of the respective needle 11, i.e., when a mesh interior space necessary for needle accommodation has been created.

As shown in FIGS. 2, 3, 4 and 5, handle part 2 also houses thread spools 29, 30, onto each of which either individual threads or a thread loop (not shown) are/is wound. The thread ends of individual threads not wound onto thread spools 29, 30 or the thread ends of the thread loop respectively wound onto thread spools 29, 30 are connected to the aforementioned needles 11. As is evident from FIGS. 8 and 9, needles 11 are relatively short and essentially oblong surgical suture needles. In FIGS. 8 and 9, the threads or thread ends are identified as 10. The respective needles 11 preferably have a relatively thin cylindrical longitudinal body made of a solid material, particularly stainless steel, with a diameter of, e.g., 0.7 mm, and a distal sharp needle tip. On the end opposite the needle tip, a thread end 10 is connected to the cylindrical longitudinal body of the respective needle 11, for example, by means of an adhesive and/or clamping connection.

In order to guide the needles 11 considered above, which are accommodated in the distal end regions of separate accommodation channels of the needle accommodation and guidance device 5, as shown in FIG. 9, out of these distal end regions and through the edge region of a tissue opening to be closed, the individual needles 11 have, in their accommodation channels (not shown), associated needle pusher members which are connected to needle pusher 4. By its displacement towards the distal end of the device, needles 11, together with the ends of suture threads 10 connected thereto, can thus be moved out of the above-mentioned distal end regions of the needle accommodation and guidance device 5 and through the edge region of a tissue opening to be closed.

The threads used in the present embodiment in the distal end of the device, with a total of four ends of two suture threads 10, are accommodated by a single thread channel 31, which is positioned, as shown in FIG. 9, in or on the circumferential region of the needle accommodation and guidance device 5. Through this thread channel 24, the respective suture threads 10 or their ends extend to their thread spools 29, 30 in the fixed handle part 2. It should be reiterated in this context, that the needle accommodation and guidance channels associated with the individual needles 11 can also be shared to accommodate the ends of suture threads 10, connected to the respective needles 11, i.e., they can be used for a combination of needle and suture thread.

As shown in FIG. 9, the individual suture threads 10 or their ends are placed in a particular way after they exit the exit opening 31 of thread channel 24. The particular manner of thread placement defines, in the case of the two thread loops wound onto the two thread spools 29, 30 in the fixed handle part 2, the manner in which the ends of suture threads 10 connected to needles 11 are guided through the edge region of a tissue opening to be closed and then retracted from the tissue opening.

FIGS. 10 to 13 illustrate four different types of thread placement with respect to a tissue opening 32 to be closed. FIGS. 10 and 11 show two cases in which the respective thread loop 33, 34 extends diagonally over the tissue opening 32 to be closed in between two respective holes which have been punctured by two needles. FIGS. 12 and 13, on the other hand, show two cases in which the respective thread loop extends between two adjacent holes 35, 36 or 37, 38, respectively created by one needle, of the tissue opening 32 to be closed and between two respective holes 35, 38 and 36, 37 or 35, 36 and 37, 38. In order to close the respective tissue opening 32, one, respectively, of the thread ends of the crossed thread loops 33, 34, as in the case shown in FIGS. 10 and 11, and one, respectively, of the thread ends of the thread loops 33, 34 running in parallel directions, as in the case shown in FIGS. 12 and 13, are pulled together and knotted. In this manner, the respective tissue opening 32 can be safely and reliably closed.

Of the suture thread ends 39, 40, 41, 42 pulled out of tissue opening 32, as shown in FIGS. 10 and 11, the one respective suture thread end 40, 41 runs underneath the respective other loop portion 34 or 33, while the respective other suture thread end 39, 42 is guided directly out of tissue opening 32 without such a course. To close tissue opening 32, in accordance with FIG. 10, the suture thread ends 39 and 40 and the suture thread ends 41 and 42 are preferably knotted together, respectively, in any particular order by means of a slip knot, or one single slip knot is formed simultaneously using all suture thread ends 39, 40, 41 and 42. In the case shown in FIG. 11, the same suture thread ends are preferably knotted with each other, respectively, as in the case shown in FIG. 10 to close tissue opening 32. For the slip knot, one suture thread end of a pair or group of suture threads, e.g. suture thread end 39, is used as the slip strand, on which a loop or knot is formed using the other suture thread of the suture thread pair or the other suture threads of the suture thread group, which loop or knot is then pushed down to the closure site of the tissue.

In accordance with FIG. 12, the thread loops 33, 34 run parallel to each other so that, here as well, the same respective suture thread ends can be pulled together and then knotted, as in the case shown in FIG. 10.

The suture thread path shown in FIG. 13 differs from the suture thread path shown in FIG. 12 only in that the suture thread ends 39 and 40 of loop portion 34 are pulled out of tissue opening 32 through the other loop portion 33. In this case as well, the same respective suture thread ends can be pulled together and knotted, as in the case shown in FIG. 10.

A further embodiment of the surgical device in accordance with the present invention will now be described in greater detail with reference to FIGS. 14 to 17.

Of the further embodiment of the surgical device in accordance with the present invention, FIGS. 14 and 15 essentially show only the open proximal area in a perspective view with a sliding linkage 25′ coupled with a needle pushing device. This sliding linkage 25′ may be pulled to the distal end of the device by means of the needle pusher 4 because a male part not shown in detail in FIG. 14 that corresponds to the male part shown in FIGS. 2, 3, 4 and 5 can be passed through a female part 17′ which exhibits an internal shape resembling the keyhole of a common case lock. In terms of function, this female part 17′ corresponds to the female part 17 shown in FIGS. 2, 3, 4 and 5.

As in the first embodiment shown in FIGS. 2, 3, 4 and 5, the female part 17′ is connected to a movement conversion device corresponding to the movement conversion device 19 provided there and hence is rotated when the rotation part 3 is rotated at the proximal end of the device.

In contrast to the first embodiment shown in FIGS. 2, 3, 4 and 5, the rotation movement of rotation part 3 according to FIG. 14 and also FIG. 15 does not act directly on the pushing part housed therein and corresponding to pushing part 14. Instead, the embodiment in accordance with FIGS. 14 and 15 provides a coupling part 46 preferably comprising two shell halves and accommodated in a fixed manner in rotation part 3. Thus, the coupling part 46 can only rotate along with rotation part 3 once it has been accommodated therein. On its inner surface, coupling part 46 is equipped with at least one journal device or journal element, as shown in FIG. 6 for the first embodiment, in order to engage in a thread of a pushing part corresponding to pushing part 14.

FIGS. 14 and 15 also show a spool receiving body 47 and a thread spool 48 positioned flat in the central handle portion of the device. Thread spool 48 houses a number of suture threads wound on their one end, with the number of threads corresponding to the number of suture needles contained in the device. Thread spool 48 may be rotated by pulling out the suture threads wound therein in the spool receiving body 47.

FIG. 15 shows a perspective view of a section of the further embodiment of the surgical device according to the invention shown in FIG. 14, along with a locking or blocking member formed by a removable safety clamp 49. This safety clamp 49 which bears the label “REMOVE”—in German “ENTFERNEN”—serves to provide additional safety for the individual handling the device. The safety clamp 49 prevents an unintentional release of the needle pusher 4 due to unintentional twisting of rotation part 3. To this end, safety clamp 49, when attached to the handle portion of the device, for example, acts along with a locking bar projecting from its inner portion or from at least one end side, on the sliding linkage 25′ and/or the female part 17′ such that, in this state, a longitudinal shift of the sliding linkage 25′ toward the distal end of the device and/or a twisting of female part 17′ and hence rotation part 3 are blocked.

It should be noted in this context that, instead of the safety clamp 49, a locking or blocking member with a different design, for example, a lever device, could be implemented in the handle portion of the device. Such a lever device, in one of its setting positions, could exert a corresponding effect on the sliding linkage 25′ and/or the female part 17′ as the safety clamp 49 when attached to the handle portion of the device. In a different setting position, the respective lever device could then release the normal device functions as with the adopted safety clamp 49.

FIG. 16 shows a considerably enlarged perspective view of a suture thread take-up device used in the further embodiment of the surgical device in accordance with the present invention shown in FIGS. 14 and 15, with the spool receiving body 47 and the thread spool 48 rotatably accommodated therein by which at least one suture thread 54 is taken up. The spool receiving body 47 has recesses 50, 51, preferably on its upper side in two diametrically opposed areas, in which the sliding linkage 25′ in accordance with FIGS. 14 and 15 is guided for a longitudinal movement after insertion of the spool receiving body 47 into the central handle portion of the device. In order to prevent twisting of the spool receiving body 47 relative to the central handle portion of the device after such insertion of the spool receiving body 47 into the central handle portion of the device, the spool receiving body 47 is received, with a rib member projecting therefrom, in a corresponding receiving groove located in the handle portion of the device, as may be seen from FIGS. 14 and 15. Other devices preventing the twisting of an element relative to another element could be used here as well.

FIG. 17 shows a perspective sectional view of the thread take-up device shown in FIG. 16 with a section below an upper cover plate of thread spool 48. As shown in FIG. 17, thread spool 48 comprises the upper cover plate, from which an inner cylinder 55 and an outer cylinder 56 project downwards. The respective suture thread 54 is wound around the inner cylinder 55, with said suture thread 54 being guided out of the spool receiving body 47 from a slot or a hole 53 in the outer cylinder 56 and through the slot 52. The aforementioned suture thread 54 may be provided either individually or in a plurality, as set forth above, and may be wound accordingly in thread spool 48. Therefore, depending on the number of suture needles present, e.g., two or four suture threads may be wound up in thread spool 48. In the event that a plurality of suture threads is taken up by the thread spool, the respective suture threads may be given the same tensions using the aforementioned slot or hole 53.

In this context, it should be noted that the thread take-up device may also have a different design than the one discussed above with reference to FIGS. 16 and 17. For example, the thread spool might exhibit only an inner cylinder and two cover plates delimiting its length, similar to a spool of a normal household sewing machine.

FIG. 18 shows a greatly enlarged sectional view of a possible design of the distal end region of device 1. It shows the distal end region of insertion tube 13 which, according to the invention, preferably houses or uses a sealing device. This sealing device serves to prevent blood from completely passing through insertion tube 13 and exiting from its proximal end during the placement of insertion tube 13 in a blood vessel through a tissue opening therein to be closed.

The sealing device comprises a liner tube 43 inserted into the insertion tube 13 on the distal side, said liner tube 43 possibly being made of a biocompatible plastic, e.g. polyamide. Inside this liner tube 43 is a sealing member 44 extending over its inner diameter, which is either initially completely closed or which can be formed, e.g., by a normally closed duckbill valve, i.e., which is closed without mechanical action. A sealing member 44 that is initially completely closed may be punctured by means of a guide wire 45 while being pushed through insertion tube 13. In the case of a sealing member 44 with a normally closed opening which is opened only by mechanical action, this opening action may be effected here via guide wire 45 when it is pushed through insertion tube 13 and sealing member 44.

The possible distal end region of device 1 shown in FIG. 18 may of course also be used in the further embodiment of the surgical device in accordance with the invention shown in FIGS. 14 and 15.

The above-described handling of the surgical device 1 according to the invention therefore includes a method for operating a surgical device designed for guiding at least one suture thread attached to a needle through the edge region of a tissue opening, particularly one present in an artery of an individual, optionally created by an incision, and for retracting the respective suture thread end from said opening after its being guided through the respective edge region. In this method, the needle connected to the respective suture thread is accommodated and guided in an accommodation and guidance opening of a rod-shaped needle accommodation and guidance device such that it is moved by a pushing device disposed at the proximal end of the device to a needle accommodation region at the distal end of the device by way of an intermediate space region, the intermediate space region in question having a length that at least corresponds to the thickness of the wall of the tissue opening, and an outer shape, with reference to the outer shape of the needle accommodation and guidance device, that is reduced for unhindered movement of the needle in question from the needle accommodation and guidance device to the distal end of the device. After having been guided through the edge region of the respective tissue opening, the respective needle is moved to the needle accommodation region, for permanent accommodation there. Then the rod-shaped needle accommodation and guidance device, along with the needle permanently accommodated in the needle accommodation region and the suture thread end connected thereto, is retracted from said tissue opening again.

In this method, the respective needle accommodated in the needle accommodation region of the rod-shaped needle accommodation and guidance device is pushed through the mesh, attached to the distal end of the device and compressible about a longitudinal body part in a longitudinal direction of the device and extendable in an opposite longitudinal direction of the device, in its compressed state, into a mesh interior space produced by the compression, and is enclosed and held tight by the mesh after such needle accommodation by subsequent extension of the mesh.

The compression and extension of the mesh are preferably accomplished by a turning or rotational movement of the aforementioned rotation part, whose rotational movement is converted to a longitudinal or translational movement.

This turning or rotational movement is preferably connected with a release/locking function for the movement/locking of the movement of the needle pusher. This ensures that the respective needle, along with the suture thread connected thereto, can only be retracted by the needle pusher from the distal end of needle accommodation and guidance device 5 after mesh 9 has been compressed, thereby forming a mesh interior space. When the surgical device 1 described is properly handled, the formation of the mesh interior space occurs within the respective tissue opening to be closed, thus ensuring the safe handling of the surgical device 1 described.

In conclusion, it should still be noted that the invention is not restricted to the embodiments described based on the drawings, but can be realized in a manner that deviates from it. Thus, it is possible according to the invention to work with a number of needles and suture thread ends connected thereto other than four, such as two or three such suture needles or, in principle, at least one needle and one suture thread attached thereto, in order to guide this suture thread by means of the associated needle through the edge region of a tissue opening, present particularly in an artery of an individual, and to retract the needle retained by the rod-shaped needle accommodation and guidance device, along with the suture thread end part connected thereto, from the tissue opening after it has been guided through it.

Furthermore, the release/blocking mechanism for the release or blocking of the displacement of the respective needle may be implemented differently than explained above. For example, the displaceability of needle pusher 4 may also be locked or unlocked, i.e., released, by a locking/unlocking pin displaceable transversely to the longitudinal direction of device 1.

The conversion device described above, serving to convert the turning or rotational movement of rotation part 3 to a longitudinal or translational movement to compress mesh 9, may also be implemented differently from the manner described above. For example, the functions of pushing part 14, which has at least one thread in its cylindrical circumference, and of pin element 22 associated with the respective thread may be interchanged in terms of a kinematic conversion. In this case, a pin element corresponding to the respective pin element 22 would be connected to the aforementioned longitudinal rod, and a female thread section, corresponding to pushing part 14, of a hollow cylindrical rotation member would be engaged with at least one female thread with the respective pin element.

### LIST OF REFERENCE CHARACTERS

1 Surgical device
2 Device handle part
3 Rotation part
4 Needle pusher
5 Needle accommodation and guidance device
6 Needle accommodation device, needle accommodation region
7 Longitudinal body component
8 Distal end part
9 Mesh
10 Suture thread
11 Needle
12 Intermediate space region
13 Insertion tube
14 Pushing part
15 Release/blocking mechanism
16 Male part
17 Female part
   17′ Female part
18 Accommodation opening, internal shape
19 Movement conversion device
20 Thread
21 Thread
22 Pin device, pin element
23 Hollow tube
24 Thread or suture thread channel
25 Hollow tube
25′ Hollow tube
26 Small guide plate
27 Support and guide post
28 Coupling mechanism
29 Thread spool
30 Thread spool
31 Exit opening
32 Tissue opening
33 Thread loop
34 Thread loop
35 Hole
36 Hole
37 Hole
38 Hole
39 Thread end
40 Thread end
41 Thread end
42 Thread end
43 Liner tube
44 Sealing member
45 Guide wire
46 Coupling part
47 Spool receiving body
48 Thread spool
49 Safety clamp
50 Recess
51 Recess
52 Slot
53 Slot
54 Suture threads
55 Inner cylinder
56 Outer cylinder

1. Surgical device (1) for guiding at least one suture thread (10) attached to a suturing needle (11) through the edge region of a tissue opening (32), particularly one present in the artery of an individual, if applicable formed by an incision, and for retracting the suture thread end guided through the edge region in question from said opening (32),
having a rod-shaped needle accommodation and guidance device (5), in which the needle (11) attached to the respective suture thread (10) is accommodated and guided in an accommodation and guidance opening such that it is movable by a pushing device (4) disposed on the proximal end of the device to a needle accommodation region (6) on the distal end of the device by way of an intermediate space region (12), said intermediate space region having a respective length corresponding at least to the thickness of the wall of the tissue opening (32), and an outer shape that is reduced relative to the outer shape of the needle accommodation and guidance device (5) in order to allow unhindered movement of the respective needle (11) from the needle accommodation and needle guidance device (5) to the distal end of the device,
the respective needle (11) being movable to the needle accommodation region (6) for permanent accommodation there after having been guided through the edge region of the tissue opening (32) in question,
and the rod-shaped needle accommodation and guidance device (5), along with the needle (11) permanently accommodated in the needle accommodation region (6), and the suture thread end connected thereto, then being retractable from the stated tissue opening (32),
wherein the needle accommodation region (6) of the rod-shaped needle accommodation and guidance device (5) is formed by a mesh (9) which is attached to the distal end of the device and compressible about a longitudinal body component (7) in a longitudinal direction of the device and extendable in an opposite longitudinal direction of the device and which, in its compressed state, allows accommodation of the respective needle (11) in a mesh interior space created by the compression and which encloses and grips the respective needle (11) by its subsequent extension after the needle has been thus accommodated.

2. Surgical device (1) according to claim 1,
wherein the respective suture thread (10) runs from a fixed device handle part (2) in a single suture thread channel (24), particularly one disposed near the exterior of the device, to the distal end region of the needle accommodation and guidance device (5) and from there to its associated needle (11).

3. Surgical device (1) according to claim 2,
wherein the needle accommodation and guidance device (5) has, for each needle (11), a separate needle accommodation and guidance channel which is provided with an entry opening near its distal end at least for the insertion of the suture thread (10) connected to the respective needle (11).

4. Surgical device (1) according claim 2,
wherein the respective needle (11) may be displaced by means of a needle pushing device (4), which is particularly displaceable on the needle accommodation and needle guidance device (5), from a position adjacent to the fixed device handle part (2) to a position towards the distal end of the device.

5. Surgical device (1) according to claim 1,
wherein the displaceability of the respective needle (11) can only be released if mesh (9) is compressed, forming the mesh interior space.

6. Surgical device (1) according to claim 2, wherein a release/blocking mechanism (15) for releasing or blocking the displacement of the respective needle (11) is disposed between the fixed device handle part (2) and a rotation device (3) adjacent thereto.

7. Surgical device (1) according to claim 6,
wherein the release/blocking mechanism (15) has a male part (16), having a specific outer shape, and a female part (17) having an inner shape (18) matching the respective outer shape such that the latter allows the accommodation of the male part (16) in a fixed rotational position of male part (16) relative to female part (17),
and in that male part (16) or female part (17) is connected to the rotation device (3) and the female part (17) or male part (16) is connected to the fixed device handle part (2).

8. Surgical device (1) according to claim 6,
wherein the rotation device (3) is connected to the fixed device handle part (2) by a conversion device (19) which converts a rotational movement into a translational movement and which has a pin device (22) that rotates along with the rotation of the rotation device (3) in the circumferential direction of the latter, and a pushing part (14) that converts its rotational movement into a longitudinal movement, said longitudinal movement making the mesh (9) compressible and thus expandable, with accompanying formation of its mesh interior space.

9. Surgical device (1) according to claim 4, wherein the rotation device (3) is positioned on the proximal end of the device adjacent to the fixed device handle part (2) and in that the needle pushing device (4) is positioned towards the distal end of the device adjacent to the fixed device handle part (2).

10. Method for operating a surgical device (1) according to claim 1 for guiding at least one suture thread (10) attached to a needle (11) through the edge region of a tissue opening (32), particularly a tissue opening present in the artery of an individual, optionally created by incision, and for retracting the respective suture thread end from the above-mentioned opening (32) after it has been guided through the respective edge region, the needle (11) connected to the respective suture thread (10) being accommodated and guided in an accommodation and guidance opening of a rod-shaped needle accommodation and guidance device (5) such that it is displaced by a pushing device (4), situated toward the proximal device end, to a needle accommodation region (6) towards the distal end of the device via an intermediate space region (12), which has a length corresponding at least to the thickness of the wall of the tissue opening (32), and an outer shape that is reduced relative to the outer shape of the needle receiving and needle guidance device (5) in order to allow unhindered movement of the respective needle (11) from the needle accommodation and guidance device (5) towards the distal end of the device,
the respective needle (11) being moved to the needle accommodation region (6), for permanent accommodation there, after having been guided through the edge region of the tissue opening (32) in question,
and the rod-shaped needle accommodation and guidance device (5), along with the needle (11) permanently accommodated in the needle accommodation region (6), and the suture thread end connected thereto, then being retracted from the above-mentioned tissue opening (32),
wherein the respective needle (11) accommodated by the needle accommodation region (6) of the rod-shaped needle accommodation and guidance device (5) is pushed through mesh (9) that is attached to the distal end of the device and is compressible about a longitudinal body component (7) in a longitudinal direction of the device and extendable in an opposite longitudinal direction of the device, when the mesh is in its compressed state, into a mesh interior space produced by the compression, and is enclosed and gripped by mesh (9) after such needle accommodation by a subsequent extension of the mesh.
